(19) United States
(12) Patent Application Publication
(10) Pub. No.: US 2022/0026367 A1
(43) Pub. Date: Jan. 27, 2022

(54) PATHOGEN SCREENING USING OPTICAL EMISSION SPECTROSCOPY (OES)

(71) Applicant: Jyotirmoy Mazumder, Ann Arbor, MI (US)

(72) Inventor: Jyotirmoy Mazumder, Ann Arbor, MI (US)

(21) Appl. No.: 17/380,270

(22) Filed: Jul. 20, 2021

Related U.S. Application Data
(60) Provisional application No. 63/054,650, filed on Jul. 21, 2020.

(51) Int. Cl. G01N 21/67; G01N 33/487; G01N 21/31

(52) U.S. Cl. CPC G01N 21/67; G01N 2201/08; G01N 21/31; G01N 33/487

(57) ABSTRACT

Apparatus and methods provide discreet and inexpensive screening for pathogens including Covid-19. A sample of bodily fluid such as saliva is energized to generate a plasma, and the optical emission spectra from the plasma is collected and analyzed used a smart optical monitoring system (SOMS) to determine the presence or increase of a protein indicative of a pathogen. The plasma may be generated with a spark, and light may be collected with a smartphone for remote analysis. In particular, in patients with Covid-19 serum concentrations of acute phase proteins (APPs), such as C-reactive protein (CRP) and ferritin, are increased in the cases that develop more severe disease. In addition, increases in serum of several interleukins (IL), such as IL-6 and IL-10, have been described in Covid-19 patients, and these cytokines are known to be mediators of the APPs response.

### REFERENCE TO RELATED APPLICATIONS

This application claims the benefit of, and priority to, U.S. Provisional Patent Application Ser. No. 63/054,650, filed Jul. 21, 2020, the entire content of which is incorporated herein by reference.

### FIELD OF THE INVENTION

This invention relates generally to pathogen screening and, in particular, to a system and method that may be used for remote screening for infectious diseases, including COVID-19 and viruses.

### BACKGROUND OF THE INVENTION

The Corona Virus (Covid19) epidemic is now affecting almost 200 countries, posing a serious threat for public health. More than 10 million people are affected world wide, resulting in more than a half-million casualties. Reliable laboratory diagnosis of the disease has been one of the foremost priorities for promoting public health interventions.

The reverse transcription polymerase chain reaction (RT-PCR) is currently the reference method for COVID-19 diagnosis[1]. However, it also reported a number of false-positive or -negative cases, especially in the early stages of the novel virus outbreak. Moreover, these types of chemical-reaction-based tests are labor, time and reagent dependent. Presently, in some areas patients have to wait for days and weeks to get test results.

Optical Emission Spectroscopy (OES) provides reagent-free, fast chemical analysis. By comparing spectra from reference sample(s) with test samples, results can be obtained in a few seconds using Smart Optical Monitoring System (SOMS)[2-5]. SOMS is described in U.S. Pat. No. 9,752,988, “In-situ identification and control of microstructures produced by phase transformation of a material,” the entire content of which is incorporated herein by reference.

With SOMS, a microstructure detector and in-situ method are used for real-time determination of the microstructure of a material undergoing alloying or other phase transformation. The method carried out by the detector includes the steps of: (a) detecting light emitted from a plasma plume created during phase transformation of a material; (b) determining at least some of the spectral content of the detected light; and (c) determining an expected microstructure of the transformed material from the determined spectral content. Closed loop control of the phase transformation process can be carried out using feedback from the detector to achieve a desired microstructure.

### SUMMARY OF THE INVENTION

This invention resides in apparatus and methods for providing discreet and inexpensive pathogen screening, including screening for the novel coronavirus Covid-19. A method of pathogen screening according to the invention includes the initial step of providing a sample of bodily fluid. In the preferred embodiments the bodily fluid is saliva. The sample is energized to generate a plasma, and the optical emission spectra from the plasma is collected and analyzed used a smart optical monitoring system (SOMS). In particular, spectra from the sample is analyzed to determine the presence or increase of a protein indicative of a pathogen in the body fluid.

The plasma may be generated by creating a spark where sample is positioned. The light from the plasma may be collected with a lens and transmitted to a SOMS system via fiber optics. The SOMS system breaks down the light into individual spectra, which is sent to a computer to analyze and provide the composition information. The light may also be collected using a camera of a smartphone, and transmitting the digitized data to a central station where the SOMS system and computer are located. Such an arrangement enables an individual to perform the test remotely (i.e., at home).

An increase in the presence of proteins in the sample can be used to diagnose patients with diseases including Covid-19. In particular, in patients with Covid-19 serum concentrations of acute phase proteins (APPs), such as C-reactive protein (CRP) and ferritin, are increased in the cases that develop more severe disease. In addition, increases in serum of several interleukins (IL), such as IL-6 and IL-10, have been described in Covid-19 patients, and these cytokines are known to be mediators of the APPs response.

Additionally, other APPs such as ferritin, haptoglobin, serum amyloid A, different interleukins, and other analytes related to the immune response, such as adenosine deaminase (ADA), can be measured in saliva. By comparing these proteins between healthy individuals and those with disease, it is possible to assess the differences, which can result from changes in the circulating levels of proteins and/or from changes in the salivary gland secretion, associated with a disease such as Covid-19.

### BRIEF DESCRIPTION OF THE DRAWINGS

FIG. 1 shows the use of a SOMS system and spark generator for analysis of a saliva sample;

FIG. 2 illustrates plasma emission spectroscopy in laser DMD; and

FIG. 3A presents a result with protein ingestion; and

FIG. 3B presents a result without protein ingestion.

### DETAILED DESCRIPTION OF THE INVENTION

Optical emission spectroscopy (OES) is a method of chemical analysis that uses the intensity of light emitted from plasma formed during material deposition to determine the quantity and quantity of elements in target objects [3-5]. In addition, the OES collection of the emission spectra generated during an additive manufacturing (AM) process can be used to provide more fundamental physical information, such as the composition of the materials. The emission signature, in addition to chemical composition, can also show the genesis of the spectrum, which can be correlated with various characteristics of the object from which plasma signal is generated.

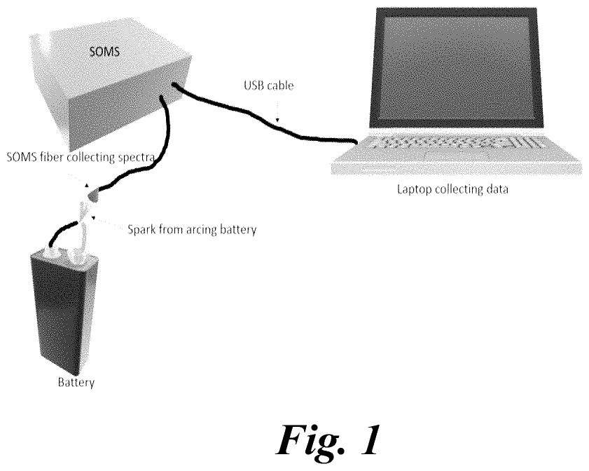

As shown in FIG. 1, plasma may be generated by creating a spark where saliva is positioned. The light from the plasma is collected by a lens and transmitted to a SOMS system via fiber optics. The SOMS system breaks down the light into individual spectra, which is sent to a computer to analyze and provide the composition information. It is possible to collect the light using a camera of a smart phone and transmit the digitized data to a central station where the SOMS system and computer are located. Such an arrangement enables an individual to perform the test remotely (i.e., at home), and send the data to a central station for analysis.

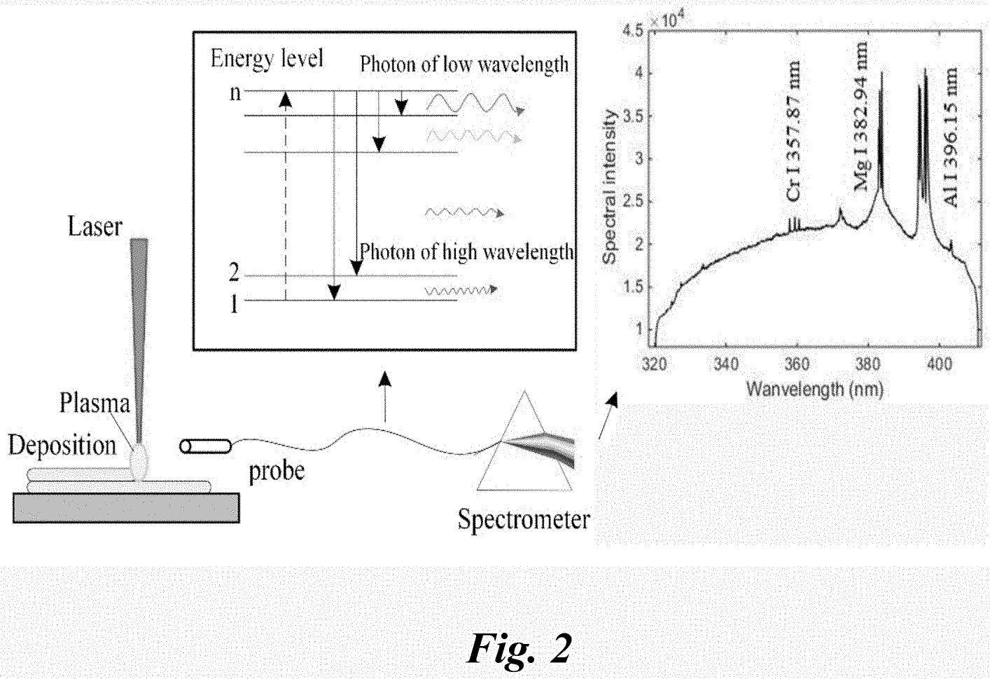

The principle of analysis of the plasma emission is illustrated in FIG. 2. The approach is similar to using a metallic additive manufacturing (AM) process. In AM, metal powders are melted and partially evaporated under the illumination of a highly energetic laser. The metal vapor and shielding gas are excited to high energy level state, with transitions to lower energy level states. In the downward electron transitions, photon wavelengths determined by the energy gaps of the transitions are released and recorded as line-emission spectra. Since the energy gaps are characteristics of elements present, the wavelengths of line emissions in spectra can be used as identifiers for the radiating elements. Further information on emission spectroscopy can be found in [6].

The spectral image in FIG. 2, for instance, is the spectra collected during laser additive manufacturing of a 7075 aluminum alloy. Peaks at wavelengths, 396.15 nm, 382.94 nm, 357.87 nm, are identified as the peaks of Al, Mg, and Cr [7] which are the main elements in the target material.

The intensity of the spectrum is proportional to the density of emitted photons. Under the local thermal equilibrium assumption, the emission density (Iᵢ_{ⱼ} (λ)) of photons is:

$${I}_{{i}_{j}} ( λ ) = \frac{1}{4 π} {n}_{0} {A}_{ij} \frac{{g}_{i} {e}^{- {E}_{i} / {k}_{B} T}}{U ( T )} I ( λ ) \# . \qquad ( 1 )$$

where the partition function U(T) is the statistical occupation fraction of every level k of the atomic species:

U(T)=Σⱼgⱼe^{-E}^{ⱼ}^{/k}^{_{B}}^{T}# (2.)

There are two types of variables associated with this analysis: 1) element-determined variables, including the wavelength of the photon (λ), the transition probability (Aᵢⱼ), the degeneracy of the upper level (gᵢ); the energy levels of level i (Eᵢ) and level j (Eⱼ); and 2) the plasma-determined variables, including the number of neutral atoms in plasma (n₀), the temperature of plasma (T), and the spectral line profile I(λ).

These variables are directly correlated with reference spectra to determine the composition and other properties. For example, the laser power density determines the temperature and electron density of the plasma, which in turn determines the intensity and profile of spectra. Parameters, including laser properties (wavelength, power distribution), powder flow rate, and shielding gas also influence the spectral properties significantly. Therefore, the relationship between spectral signal and manufacturing quality means OES has significant potential for in-situ diagnosis.

### Preliminary Results for Protein Identification:

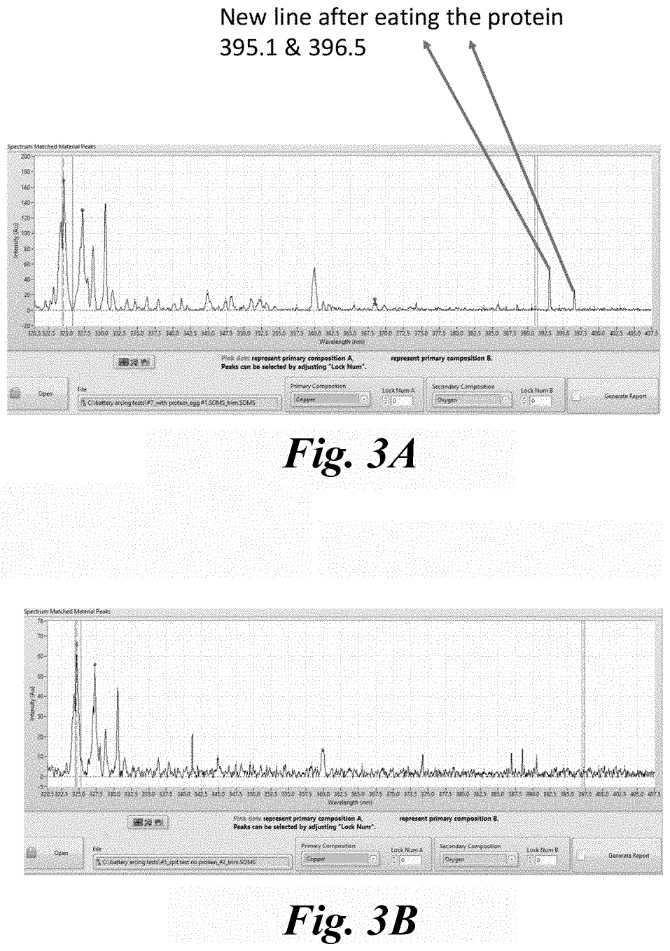

FIGS. 3A, B show the spectra of saliva with and without protein. An increase in the presence of proteins in saliva can be used to diagnose patients with Covid-19. In particular, in patients with Covid-19 serum concentrations of acute phase proteins (APPs), such as C-reactive protein (CRP) and ferritin, are increased in the cases that develop more severe disease. In addition, increases in serum of several interleukins (IL), such as IL-6 and IL-10, have been described in Covid-19 patients, and these cytokines are known to be mediators of the APPs response.

Additionally, other APPs such as ferritin, haptoglobin, serum amyloid A, different interleukins, and other analytes related to the immune response, such as adenosine deaminase (ADA), can be measured in saliva. By comparing these proteins between healthy individuals and those with disease, it is possible to assess the differences, which can result from changes in the circulating levels of proteins and/or from changes in the salivary gland secretion, associated with a disease such as Covid-19.

### REFERENCES

1) Guangyu Qiu, Zhibo Gai, Yile Tao, Jean Schmitt, Gerd A. Kullak-Ublick, and Jing Wang; Dual-Functional Plasmonic Photothermal Biosensors for Highly Accurate Severe Acute Respiratory Syndrome Coronavirus 2 Detection; ACSNano; https://dx.doi.org/10.1021/acsnano.0c02439, May 20, 2020
2) U.S. Pat. No. 9,752,988, In-situ identification and control of microstructures produced by phase transformation of a material, Inventor; Jyoti Mazumder eta. al, Sep. 5, 2017
3) J. Mazumder, “Design for Metallic Additive Manufacturing Machine with Capability for “Certify as You build”,” in Cirp 25th Design Conference Innovative Product Creation, vol. 36, 2015, pp. 187-192.
4) L. Song, W. Huang, X. Han, and J. Mazumder, “Real-Time Composition Monitoring Using Support Vector Regression of Laser-Induced Plasma for Laser Additive Manufacturing,” Ieee Transactions on Industrial Electronics, vol. 64, no. 1, pp. 633-642, January 2017.
5) C. B. Stutzman, A. R. Nassar, and E. W. Reutzel, “Multi-sensor investigations of optical emissions and their relations to directed energy deposition processes and quality,” (in English), Additive Manufacturing, Article vol. 21, pp. 333-339, May 2018.
6) U. Fantz, “Basics of plasma spectroscopy,” (in English), Plasma Sources Science & Technology, vol. 15, no. 4, pp. 5137-5147, November 2006.
7) Y. Ralchenko, A. Kramida, J. J. N. I. o. S. Reader, and G. Technology, MD, “NIST atomic spectra database,” 2008.

1. A method of pathogen screening, comprising the steps of:
providing a sample of body fluid;
delivering energy to the sample sufficient to generate a plasma;
collecting optical emission spectra from the plasma; and
analyzing the optical emission spectra to determine the presence or increase of a protein indicative of a pathogen in the body fluid.

2. The method of claim 1, wherein the pathogen is a bacterium, virus, or other microorganism that can cause disease.

3. The method of claim 2, wherein the pathogen is a coronavirus.

4. The method of claim 1, wherein the protein is an acute phase protein (APP) or APP mediator.

5. The method of claim 1, wherein the protein is a C-reactive protein (CRP).

6. The method of claim 1 wherein the protein is ferritin.

7. The method of claim 1 wherein the protein is haptoglobin.

8. The method of claim 1 wherein the protein is amyloid A.

9. The method of claim 1 wherein the protein is an analytes related to an immune response.

10. The method of claim 1 wherein the protein is adenosine deaminase (ADA).

11. The method of claim 1, wherein the protein is an interleukin (IL).

12. The method of claim 10, wherein the interleukin is IL-6 or IL-10.

13. The method of claim 4, wherein the protein is a cytokine.

14. The method of claim 1, wherein the sample of body fluid contains saliva.

15. The method of claim 1, wherein the energy delivered to the sample sufficient to generate a plasma is produced with an electrical spark.

16. The method of claim 1, wherein the optical emission spectra from the plasma is delivered to a smart optical monitoring system (SOMS) via an optical fiber.

17. The method of claim 1, wherein the plasma is using a camera of a smart phone and transmitted as digitized data to a remote SOMS system for analysis.
